(19) United States
(12) Patent Application Publication
(10) Pub. No.: US 2014/0180134 A1
(43) Pub. Date: Jun. 26, 2014

(54) IMAGING CATHETER FOR IMAGING FROM WITHIN BALLOON

(71) Applicant: VOLCANO CORPORATION, San Diego, CA (US)

(72) Inventor: Paul Hoseit, El Dorado Hills, CA (US)

(73) Assignee: VOLCANO CORPORATION, San Diego, CA (US)

(21) Appl. No.: 14/108,627

(22) Filed: Dec. 17, 2013

Related U.S. Application Data
(60) Provisional application No. 61/740,479, filed on Dec. 21, 2012.

(51) Int. Cl. A61B 5/00

(52) U.S. Cl. CPC A61B 5/0084; A61B 5/0095
USPC 600/478; 600/407

(57) ABSTRACT

The invention generally relates to balloon catheters for vascular intervention and particularly to devices for imaging from within a balloon. The invention provides a balloon catheter with an imaging device inside the balloon and capable of viewing a treatment site through a wall of the balloon. The device allows a physician to both view the affected site within the vessel and to inflate the balloon at the location that is in view, thus allowing the balloon to be deployed with good positioning and efficiency while minimizing a stiff length of the catheter to give it good maneuverability.

### CROSS-REFERENCE TO RELATED APPLICATION

This application claims the benefit of, and priority to, U.S. Provisional Patent Application Ser. No. 61/740,479, filed Dec. 21, 2012, the contents of which are incorporated by reference.

### FIELD OF THE INVENTION

The invention generally relates to balloon catheters for vascular intervention and particularly to devices for imaging from within a balloon.

### BACKGROUND

Atherosclerosis, or hardened arteries, involves the buildup of plaque inside blood vessels. The buildup of plaque restricts the flow of blood, and thus nutrients and oxygen, to a person's tissue and brain. Sometimes chunks of the atherosclerotic plaque break away and flow through the person's blood vessels. This can lead to serious and deadly strokes and heart attacks.

Vascular balloon catheters are one tool for treating atherosclerosis. In a treatment known as balloon angioplasty, a catheter is used to inflate a balloon within the narrowed vessel to crush the plaque and open up the vessel. The balloon is then withdrawn, allowing blood to flow freely. The balloon may also be used to implant a stent to support the newly opened vessel.

Blood vessels have countless forks and turns, none of which are visible to the naked eye. Nevertheless, angioplasty requires maneuvering the catheter to the affected area and using the balloon in the right spot. Even though some catheters have imaging devices, maneuverability and visibility are significant problems. For example, each device on a catheter tends to stiffen the catheter and decrease its flexibility. Thus, adding an ultrasonic imaging probe near a balloon or stent interferes with maneuverability. Moreover, deploying the balloon in the correct location can require multiple iterations of viewing the affected site, sliding the catheter into position, inflating the balloon, pulling the catheter back to look again, and repeating. This trial-and-error approach requires the patient to have a catheter threaded into their veins for a prolonged time, which aggravates the patient's discomfort, as well as increasing costs and risks of complications.

### SUMMARY

The invention provides a balloon catheter with an imaging device inside the balloon and capable of viewing a treatment site through a wall of the balloon. Since this arrangement allows a physician to both view the affected site within the vessel and to inflate the balloon at the location that is in view, the device allows a balloon to be deployed in just the right location with a single inflation. Locating the imaging device inside of the balloon also minimizes a stiff length of the catheter. Due to its increased flexibility, the catheter is more maneuverable, and a doctor can more readily position the balloon properly at the treatment site. Since the doctor can view the treatment site directly through the balloon and deploy the balloon in the correct location, multiple iterations of catheter positioning are avoided. Since the balloon can be maneuvered to the correct location and deployed with precision and accuracy, treatment does not require a prolonged amount of time. Thus, patient discomfort and unnecessary costs as well as high risks of complications are all avoided. With these tools, more patients can be treated for atherosclerotic conditions that would otherwise pose a significant risk of stroke and heart attack.

In certain aspects, the invention provides a vascular balloon catheter generally having an elongate shaft with a proximal portion and a distal portion and having an inflatable balloon disposed at the distal portion for insertion into a vessel. An image detector is disposed within the balloon to take an image of the vessel and treatment site by receiving a signal through the balloon. The image detector can be located on the surface of the elongate shaft of the catheter and the elongate shaft can provide a guidewire lumen for angioplastic guidewire procedures. The image detector may include a fiber that is on an exterior of the elongate member within the balloon and entirely within the elongate shaft everywhere outside of the balloon. In some embodiments, the image detector uses an optical fiber, a photoacoustic transducer, or both. For example, the image detector can include a fiber Bragg grating. This can be used with a photoacoustic transducer to receive a signal through the balloon as sound and to send the signal from the balloon to the proximal portion of the catheter (e.g., along the optical fiber) as light. Where the image detector employs an optoacoustic imaging modality, acoustic energy may propagate substantially perpendicular to an axis of the catheter and light may propagate substantially parallel to the axis.

By using an optical fiber, positioning the image detector within the balloon, or both, a catheter may be provided that has imaging capabilities and also a substantially uniform flexibility everywhere along its length outside of the balloon.

In related aspects, the invention provides a method of delivering an angioplasty balloon by using an elongate catheter having a balloon disposed at a distal portion of the catheter to deliver the balloon to a treatment site within a vessel. The treatment site is viewed from within the balloon using an image detector on the distal portion within the balloon. An operator may decide when and where to inflate the balloon based on viewing the treatment site. Inflating the balloon causes an exterior surface of the balloon to make contact with the treatment site and dilate the vessel. Methods of the invention may also optionally be used to deliver and deploy a stent.

In some embodiments, the treatment site is viewed via ultrasound imaging technology, optical-acoustical imaging, or other suitable methods. For example, an ultrasound image signal may be received at the image detector and converted into an optical interferometric signal using the image detector. The image detector may employ one or more of an optical fiber; a fiber Bragg grating; a blazed fiber Bragg grating; photoacoustic transducer; other elements; or a combination thereof.

### BRIEF DESCRIPTION OF THE DRAWINGS

FIG. 1 shows a catheter according to certain embodiments of the invention.

FIG. 2 gives a cross-sectional view through a distal portion of the catheter.

FIG. 3 diagrams an imaging fiber of the invention.

FIG. 4 illustrates a catheter with an inflated balloon with imaging devices therein.

FIG. 5 shows a cross-section of the device shown in FIG. 4.

FIG. 6 shows carrying a balloon towards a feature of interest.

FIG. 7 shows imaging through an un-inflated balloon.

FIG. 8 shows imaging through an inflated balloon.

FIG. 9 shows a catheter and stent according to some embodiments.

FIG. 10 is a cross section along the dotted line in FIG. 9.

FIG. 11 depicts a catheter, balloon, and stent with a plurality of imaging devices.

FIG. 12 presents a cross sectional view along the dotted line in FIG. 11.

FIG. 13 shows a perspective view of a device according to certain embodiments.

### DETAILED DESCRIPTION

The invention generally relates to intravascular balloon catheters, and more particularly to a balloon catheter that provide the ability to capture an image from within the balloon.

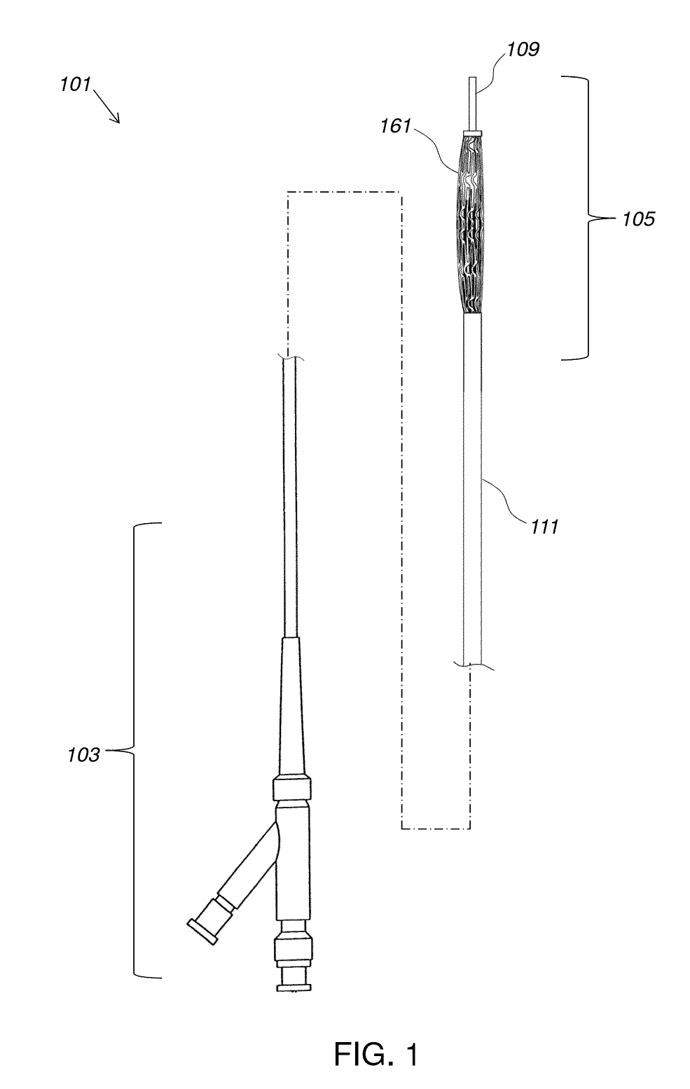

FIG. 1 shows a catheter 101 according to certain embodiments of the invention. Catheter 101 includes a proximal portion 103 that is generally outside of a patient during use and a distal portion 105 extending to a distal tip 109 configured for insertion into a patient. Distal portion 105 may generally include a treatment device. Pictured in FIG. 1 is a stent 161 disposed around a balloon, but any suitable treatment device may be included. A length of catheter 101 extending through distal portion 105 generally defines a catheter shaft 111 capable of being delivered over a guidewire (guidewire not pictured). Intravascular balloon catheters are used for such procedures as balloon angioplasty, or percutaneous transluminal coronary angioplasty (PTCA). A catheter generally has an elongate tubular shaft 111 with proximal portion 103 and distal portion 105, and may include one or more passages or lumens. Use of pliable materials provides flexibility or maneuverability, allowing a catheter to be guided to a treatment site in a patient's blood vessels. Preferably, a catheter of the invention has enough stiffness to allow it to be pushed to a target treatment site, and accordingly, an ability to optimize a balance of pliability versus stiffness or pushability is beneficial to medical use. Moreover, a shaft of the catheter can be provided that is capable of transmitting torque along an axis of the shaft. Devices for cardiovascular intervention are discussed in U.S. Pat. Nos. 6,830,559; 6,074,362; and U.S. Pat. No. 5,814,061, the contents of each of which are incorporated by reference.

Catheter 101 includes an angioplasty balloon 107 or other interventional device at distal portion 105 to expand or dilate blockages in blood vessels or to aid in the delivery of stents or other treatment devices. Blockages include the narrowing of the blood vessel called stenosis.

Typically, elongate shaft 111 of catheter 101 will include a guidewire lumen so that the catheter may be advanced along a guidewire. Guidewire lumen in a balloon catheter is described in U.S. Pat. No. 6,022,319 to Willard. Elongate shaft 111 may include any suitable material such as, for example, nylon, low density polyethylene, polyurethane, or polyethylene terephthalate (PET), or a combination thereof (e.g., layers or composites). An inner surface of a guidewire lumen may include features such as a silicone resin or coating or a separate inner tube made, for example, of preformed polytetrafluoroethylene (PTFE). The PTFE tube may be installed within the catheter shaft by sliding it into place and then shrinking the catheter shaft around it. This inner PTFE sleeve provides good friction characteristics to the guidewire lumen, while the balance of the catheter shaft can provide other desired qualities. Other suitable materials for use in catheter 101 or an inner tube portion thereof include high density polyethylene (HDPE) or combinations of material, for example, bonded in multiple layers.

Catheter 101 may include coaxial tubes defining separate inflation and guidewire lumens, for example, along a portion of, or an entirety of, a length of catheter 101. A plurality of lumens may be provided in parallel configuration or coaxial at one point and parallel at another, with a twisting/plunging portion to affect a transition between the parallel segment and the coaxial segment (see., e.g., U.S. Pat. No. 7,044,964). Other possible configurations include one or more of a guidewire tube or guidewire lumen disposed outside of the balloon. Or the guidewire tube may be affixed to and extend along the wall of the balloon.

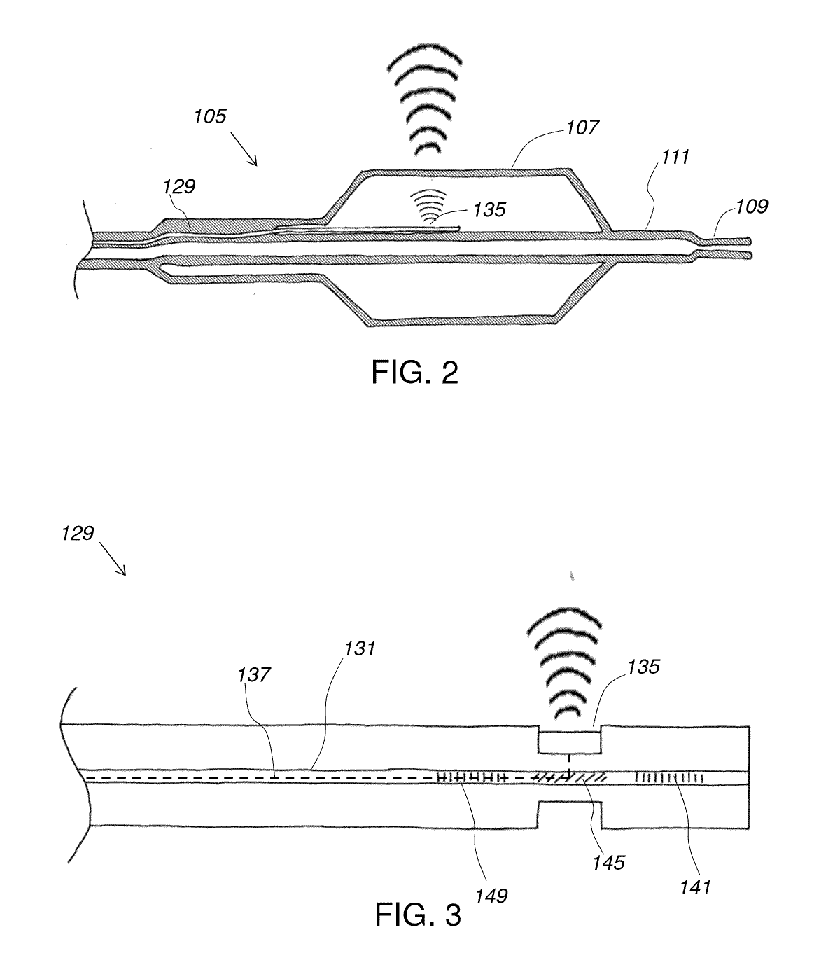

FIG. 2 shows a cross section of distal portion 105 of catheter 101. Disposed on a surface of shaft 111 is imaging device 135. As shown in FIG. 2, imaging device 135 is within balloon 107. At distal tip 109 an opening into catheter 101 can be seen, allowing distal portion 105 of catheter 101 to be slid over a guidewire. Balloon 107 may include any suitable material. Generally, balloon 107 will include a flexible, inelastic material designed to expand. By this type of expansion, a balloon may impose pressures of several atmospheres to expand the stenosis or may be used to deploy a stent. After the balloon has been expanded, it is then deflated and removed from the patient, allowing improved blood flow through the vessel. Suitable materials may include polyvinyl chloride (PVC), nylon, polyethylene terephthalate (PET), polybutylene terephthalate (PBT) and copolyesters, polyether-polyester block copolymers, polyamides, polyurethane, poly(ether-block-amide) and the like. Balloons are described in U.S. Pat. No. 7,004,963; U.S. Pub. 2012/0071823; U.S. Pat. No. 5,820,594; and U.S. Pub. 2008/0124495, the contents of each of which are incorporated by reference. Balloon catheters are described in U.S. Pat. No. 5,779,731 and U.S. Pat. No. 5,411,016, incorporated by reference.

In some embodiments, the balloon includes artificial muscle (electro-active polymer). Electro-active polymers exhibit an ability to change dimension in response to electric stimulation. The change may be driven by electric field E or by ions. Exemplary polymers that respond to electric fields include ferroelectric polymers (commonly known polyvinylidene fluoride and nylon 11, for example), dielectric EAPs, electro-restrictive polymers such as the electro-restrictive graft elastomers and electro-viscoelastic elastomers, and liquid crystal elastomer composite materials. Ion responsive polymers include ionic polymer gels, ionomeric polymer-metal composites, conductive polymers and carbon nanotube composites. Common polymer materials such as polyethylene, polystyrene, polypropylene, etc., can be made conductive by including conductive fillers to the polymer to create current-carrying paths. Many such polymers are thermoplastic, but thermosetting materials such as epoxies, may also be employed. Suitable conductive fillers include metals and carbon, e.g., in the form of sputter coatings. Electro-active polymers are discussed in U.S. Pat. No. 7,951,186; U.S. Pat. No. 7,777,399; and U.S. Pub. 2007/0247033, the contents of each of which are incorporated by reference.

As shown in FIG. 2, imaging device 135 is positioned near the end of an imaging fiber 129. A substantial length of imaging fiber 129 extends within catheter shaft 111 and may be embedded in a material of the shaft or located within a lumen (e.g., a dedicated lumen or a shared lumen). In some embodiments, an entirety of imaging fiber 129 extends along a surface of catheter shaft 111. Thus, the detection element 135 is located within the lumen of balloon 107 (i.e., an intraluminal detection element) and configured to image directly at the therapy site. Accordingly, no further repositioning of the device is required after deployment of balloon 107.

Imaging device 135 can employ any suitable imaging modality known in the art. Suitable imaging modalities include intravascular ultrasound (IVUS), optical coherence tomography (OCT), optical-acoustical imaging, and others. For ultrasound imaging, catheter 101 may include an ultrasound transducer as imaging device 135. Ultrasonic imaging catheters are discussed in U.S. Pat. No. 5,054,492 to Scribner; U.S. Pat. No. 5,024,234 to Leary; and U.S. Pat. No. 4,841,977 to Griffith. Systems for IVUS are discussed in U.S. Pat. No. 5,771,895; U.S. Pub. 2009/0284332; U.S. Pub. 2009/0195514; U.S. Pub. 2007/0232933; and U.S. Pub. 2005/0249391, the contents of each of which are hereby incorporated by reference in their entirety. OCT systems and methods are described in U.S. Pub. 2011/0152771; U.S. Pub. 2010/0220334; U.S. Pub. 2009/0043191; U.S. Pub. 2008/0291463; and U.S. Pub. 2008/0180683, the contents of each of which are hereby incorporated by reference in their entirety. In certain embodiments, catheter 101 makes use of a combination of optical and acoustic signal propagation for imaging capabilities.

FIG. 3 shows an imaging fiber 129 that allows for an optical-acoustic imaging technique. In operation, light 137 is transmitted along fiber optic core 131. In some embodiments, fiber optical core 131 includes one or more fiber Bragg grating 149, 141, or others. When light 137 reaches fiber Bragg grating 149, some of it is reflected back to the proximal end of imaging fiber 129 and some of light 137 passes to a distal side of fiber Bragg grating 149. Fiber optic core 131 may include one or more of a blazed fiber Bragg grating 145 that reflects light 137 in a direction that is substantially perpendicular to an axis of imaging fiber 129. The perpendicular light 137 then impinges on photoacoustic transducer 135. The light energy is converted into phonons of heat which cause the expansion of photoacoustic transducer 135. As photoacoustic transducer 135 expands in pulses synchronous with incoming pulses of light, an exterior surface of transducer 135 initiates a longitudinal wave (e.g., a sound wave) that propagates away from imaging fiber 129 through a patient's blood and tissue (or any other fluids and materials). This ultrasonic energy interrogates the tissue—bouncing off of vessel walls and other features—and returns an ultrasonic image. The returning ultrasonic signal can be transduced onto an optical carrier signal through the use of the same photoacoustic transducer 135 or a different one.

Light reflected by blazed fiber Bragg grating 145 from photoacoustic transducer 135 and into fiber core 131 combines with light that is reflected by either fiber Bragg grating 149 or 141 (either or both may be including in various embodiments). The light from photoacoustic transducer 135 will interfere with light reflected by either fiber Bragg grating 149 or 141 and the light 137 returning to the control unit will exhibit an interference pattern. This interference pattern encodes the ultrasonic image captured by imaging device 135. The light 137 can be received into photodiodes within a control unit and the interference pattern thus converted into an analog electric signal. This signal can then be digitized using known digital acquisition technologies and processed, stored, or displayed as an image of the target treatment site. An incoming optical acoustical signal impinging on diodes creates an analog electrical signal which can be digitized according to known methods. Methods of digitizing an imaging signal are discussed in Smith, 1997, THE SCIENTIST AND ENGINEER'S GUIDE TO DIGITAL SIGNAL PROCESSING, California Technical Publishing (San Diego, Calif.), 626 pages; U.S. Pat. No. 8,052,605; U.S. Pat. No. 6,152,878; U.S. Pat. No. 6,152,877; U.S. Pat. No. 6,095,976; U.S. Pub. 2012/0130247; and U.S. Pub. 2010/0234736, the contents of each of which are incorporated by reference for all purposes.

In some embodiments, imaging fiber 129 operates to receive the incoming ultrasonic signal without necessarily being the source of the outgoing ultrasonic signal. An outgoing ultrasonic signal may be provided by a neighboring transducer 135, by another ultrasonic transducer such as a guidewire transducer, or by using balloon 107 itself as the source of ultrasonic energy. Angioplasty balloons as a source of ultrasonic excitation are discussed in U.S. Pat. No. 6,398,792 to O'Connor; U.S. Pat. No. 5,609,606 to O'Boyle. While using image detector 135 to view the target tissue, an operator can position balloon 107 in the appropriate place and inflate it.

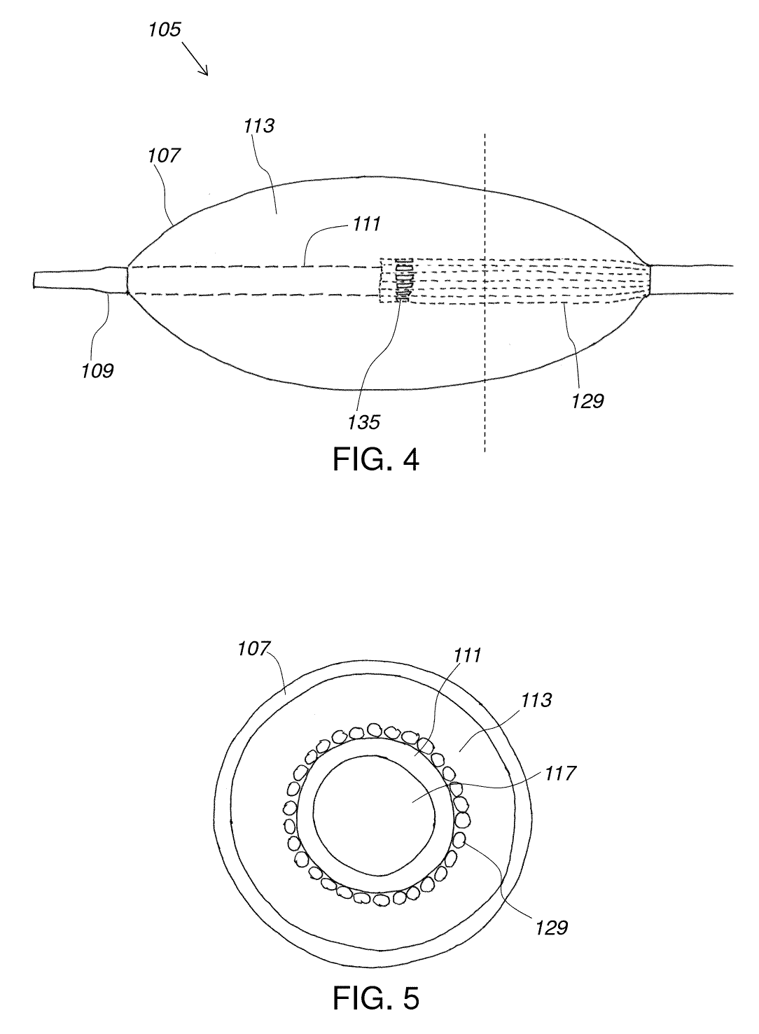

FIG. 4 illustrates a proximal portion 105 of catheter 101 with an inflated balloon 107 with a plurality of imaging fibers 129 therein. Comparing FIG. 4 to FIG. 2, it can be seen that catheter 101 can include one or any number of imaging fibers 129. In some embodiments, catheter 101 includes 2, 3, 4, 5, 6, or more imaging fibers (e.g., 10, 12, 15, 16, 32, 35, 30, 50, 64, 75, 100, hundreds, etc.). Each imaging fiber can include one or a number of image detectors 135 (e.g., 2, 3, 4, 5, etc.). Image detectors 135 may be disposed substantially within an area of a plane that is substantially perpendicular to an axis of catheter 111, as shown in FIG. 4, or they may be arrayed in other patterns, e.g., displaced from one another to define an helix around catheter 101 or spaced irregularly, etc.

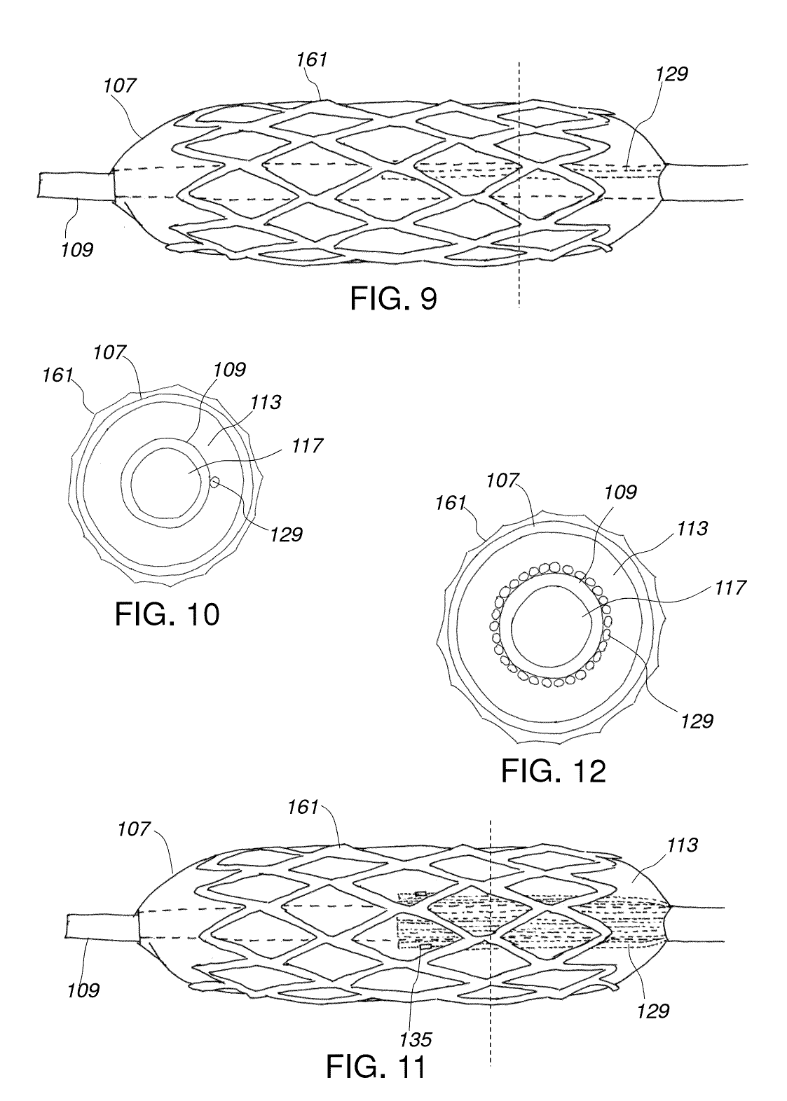

FIG. 5 shows a cross-section of the device through the dotted line shown in FIG. 4 illustrating that elongate shaft 111 may define a guidewire lumen 117 therein. One or a plurality of imaging fibers 129 may be disposed on a surface of elongate shaft 111. Around a body of elongate shaft 111 is balloon 107, spaced away by inflation lumen 113 (although in a deflated state, balloon 107 may have any geometry, such as an irregular shape, and may be substantially compressed against a body of elongate shaft 111). Disposed on a surface of elongate shaft 111 are a plurality of imaging fibers 129. Each imaging fiber 129 presents an image detector 135 facing substantially away from an axis of elongate shaft 111. As shown in FIGS. 9-12, optional stent 161 may be disposed around an outside of balloon 107.

The invention includes methods of providing an array of imaging fibers 129 that can be disposed around elongate shaft 111 as shown in FIG. 5 and further provides methods of creating a plurality of image detectors 135 that are all oriented in a desired direction. In some embodiments, a plurality of substantially featureless optical fibers are arrayed in a sheet substantially parallel to one another. The sheet of fibers may be positioned on a sheet of material that may optionally have an adhesive on the surface. Additionally or alternatively, a cementing material may be applied to the sheet-like array of fibers. The fibers 129 may be arrayed in substantially straight lines (e.g., by combing prior to application of adhesive or cement) or may be in other conformations. For example, introducing a wavy or zigzag pattern into a portion of the fibers 129 may give them slack, or “play”, that allows image detectors to stay in place on a surface of balloon 107 when balloon 107 is inflated. Once the fibers are so arrayed and held in place, the fiber Bragg gratings may then be formed in all of them. The fiber Bragg gratings may be formed by an inscribing method using a UV laser and may be positioned through the use of interference or masking. Inscribing and use of fiber Bragg gratings are discussed in Kashyap, 1999, FIBER BRAGG GRATINGS, Academic Press (San Diego, Calif.) 458 pages; Othonos, 1999, FIBER BRAGG GRATINGS: FUNDAMENTALS AND APPLICATIONS IN TELECOMMUNICATIONS AND SENSING, Artech (Norwood, Mass.) 433 pages; U.S. Pat. No. 8,301,000; U.S. Pat. No. 7,952,719; U.S. Pat. No. 7,660,492; U.S. Pat. No. 7,171,078; U.S. Pat. No. 6,832,024; U.S. Pat. No. 6,701,044; U.S. Pub. 2012/0238869; and U.S. Pub. 2002/0069676, the contents of each of which are incorporated by reference.

Detectors 135 can then be introduced by grinding a channel into the surface of all of the fibers. If done with the fibers un-cemented, the fibers can be rolled over and the grinding continued so that each fiber has an annular channel extending around the fiber. Fiber Bragg grating 149, 141, both, others, or a combination thereof can be formed, as well as any desired number of blazed fiber Bragg grating 145 in each fiber 129. A channel or cutaway can be formed for image detector and may optionally be filled with a photoacoustic transducer material. Suitable photoacoustic materials can be provided by polydimethylsiloxane (PDMS) materials such as PDMS materials that include carbon black or toluene. Imaging fibers and methods of making them are discussed in U.S. Pat. No. 8,059,923, the contents of which are incorporated by reference for all purposes. Once the sheet-like array is bound together (e.g., the adhesive has set), the sheet can be applied to a surface—for example, wrapped around catheter shaft 111.

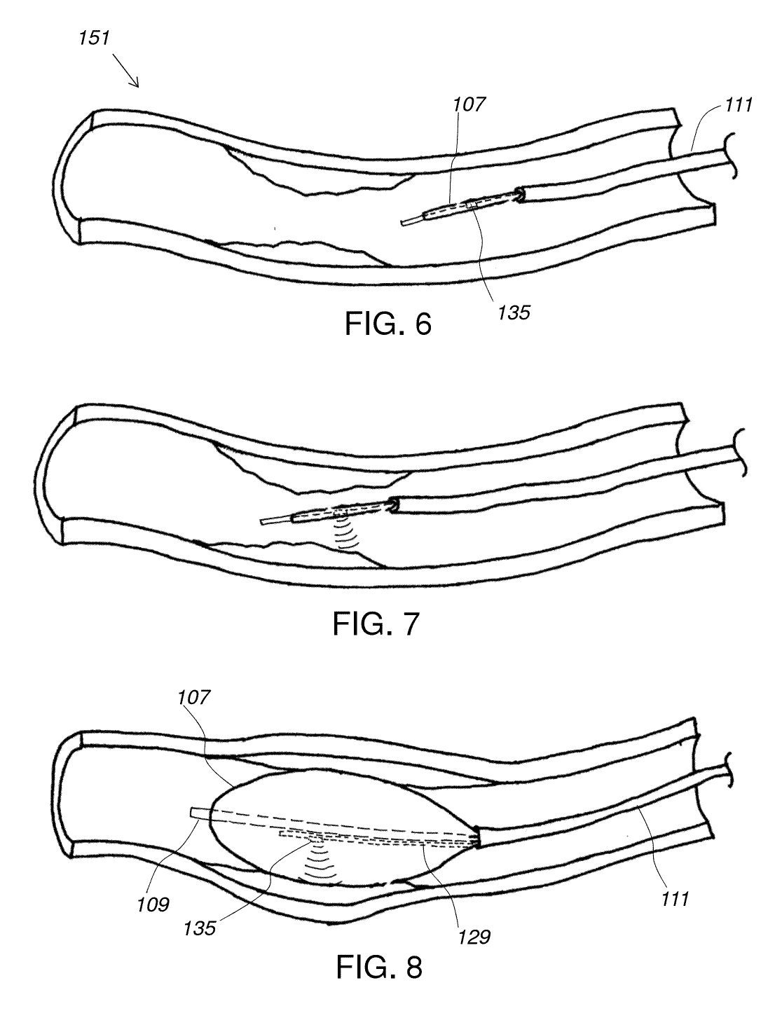

FIGS. 6-8 show use of balloon 107 with imaging fiber 129 and image detector 135 therein to view a treatment site 151. As distal portion 105 of catheter 101 approaches treatment site 151 (such as a region of a blood vessel affected by atherosclerotic plaque), a physician can view site 151 on a monitor of an associated medical imaging instrument (not pictured). Using, for example, IVUS or optical-acoustic imaging, the vessel wall is viewed to monitor for the location of atherosclerotic plaques. Monitoring a position of catheter 101 may also be optionally combined with use of standard x-ray angiographic techniques. When balloon 107 is positioned at the target treatment site, it is inflated, as shown in FIG. 8, thus opening a passageway that will allow blood to flow past the stenosized (narrowed) portion of the vessel after the balloon is deflated. Balloon 107 may also be optionally used to deploy a stent. Such vascular intervention procedures by catheter are often performed in specialized clinical environments known as cath labs. Cath labs and associated imaging instrumentation (e.g., IVUS and OCT instruments) are known in the art. For example, IVUS is discussed in U.S. Pat. No. 8,289,284; U.S. Pat. No. 7,773,792; U.S. Pub. 2012/0271170; U.S. Pub. 2012/0265077; U.S. Pub. 2012/0226153; and U.S. Pub. 2012/0220865. OCT systems and methods are described in U.S. Pub. 2011/0152771; U.S. Pub. 2010/0220334; U.S. Pub. 2009/0043191; U.S. Pub. 2008/0291463; and U.S. Pub. 2008/0180683, the contents of each of which are hereby incorporated by reference in their entirety. Optical-acoustic imaging structures (e.g., for imaging fiber 129) are discussed in U.S. Pat. No. 8,059,923; U.S. Pat. No. 7,660,492; U.S. Pat. No. 7,527,594; U.S. Pat. No. 6,261,246; U.S. Pat. No. 5,997,523; U.S. Pub. 2012/0271170 and U.S. Pub. 2008/0119739. The contents of each of these patents and publications are incorporated by reference in their entirety for all of their teachings and for all purposes.

Use of a catheter 101 of the invention allows for imaging from within a balloon and this may aid in properly delivering and positioning a stent 161.

FIG. 9 shows a proximal portion 105 of catheter 101 with stent 161 on balloon 107. Any suitable stent 161 may be used with device 101. One exemplary device for stent 161 is the Palmaz-Schatz stent, described, for example, in U.S. Pat. No. 4,733,665. Suitable stents are described in U.S. Pat. No. 7,491,226; U.S. Pat. No. No. 5,405,377; U.S. Pat. No. 5,397,355; and U.S. Pub. 2012/0136427, the contents of each of which are expressly incorporated herein by reference. Generally, stent 161 has a tubular body including a number of intersecting elongate struts. The struts may intersect one another along the tubular body. In a non-deployed state, the tubular body has a first diameter that allows for delivery of stent 161 into a lumen of a body passageway. When deployed, stent 161 has a second diameter and deployment of stent 161 causes it to exert a radially expansive force on the lumen wall. Methods of using stents are discussed in U.S. Pat. 6,074,362; U.S. Pat. No. 5,158,548; and U.S. Pat. No. 5,257,974, the contents of each of which are incorporated by reference. In some embodiments, stent 161 includes a shape-retaining or shape memory material such as nitinol and is self-expanding and thermally activatable within a vessel upon release. Such devices may automatically expand to a second, expanded diameter upon being released from a restraint. See, e.g., U.S. Pat. No. 5,224,953, the contents of which are incorporated herein by reference.

FIG. 10 gives a cross section along the dotted line in FIG. 9. As shown in FIGS. 9 and 10, imaging fiber 129 is positioned to “see through” balloon 107 and stent 161. Image detector 135 may be specifically located to detect an image through an aperture of stent 161 but more preferably, a material of stent 161 is functionally transparent or translucent to a modality of imaging employed by imaging element 135. For example, in some embodiments, where imaging element 135 operates by ultrasound or optical-acoustical ultrasound, an ultrasonic signal may propagate through stent 161, thereby detecting both stent 161 itself as well as bodily fluids and tissues around stent 161. FIGS. 9 and 10 illustrate an embodiment in which a single imaging fiber 129 is disposed on a surface of elongate shaft 111. However, any number of fibers may be included.

FIG. 11 depicts proximal portion 105 of catheter 101 having a plurality of imaging fibers 129 surrounding elongate shaft 111. While shown in FIG. 11 as lying substantially against one another, imaging fibers 129 may be spaced apart or may be overlapping, including overlapping so much as to define multiple layers. Considerations of geometries of a surface of balloon and changes thereto during inflation and de-inflation of balloon 107 may inform the positioning of imaging fibers 129. For example, a portion of any or all of fibers 129 may be slack or zigzag shaped to allow give during inflation. A multitude of fibers 129 may be provided at one density to achieve a desired density after inflation. As balloon 107 inflates, a circumference of balloon 107 changes by a factor of the square of the radius of balloon 107. Thus it may provide beneficial multi-directional viewing to provide a plurality of fibers 129 that at least touch or even overlap one another.

FIG. 12 presents a cross sectional view along the dotted line in FIG. 11. Here, fibers 129 abut one another.

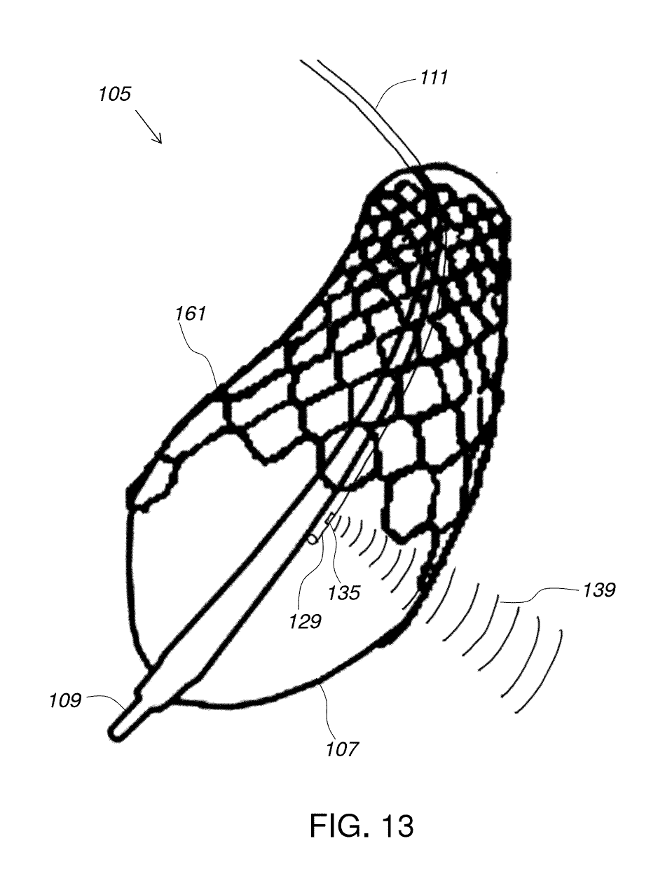

FIG. 13 gives a perspective view of catheter 101 as depicted in FIG. 1 in a deployed state. Balloon 107 has been inflated via inflation lumen 119. Stent 161 has been expanded. If inside of a vessel at treatment site 151, stent 161 will then remain in place when balloon 107 is deflated and catheter 111 is withdrawn and removed from the patient. FIG. 13 depicts a single imaging fiber 129 extending within balloon 107. This arrangement is provided and may be desired in some embodiments.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. An intravascular imaging catheter comprising:
an elongate shaft having a proximal portion and a distal portion;
an inflatable balloon disposed at the distal portion for insertion into a vessel; and
an image detector disposed within the balloon and configured to receive a signal through the balloon, the signal comprising an image of the vessel.

2. The catheter of claim 1, wherein the image detector comprises an optical fiber.

3. The catheter of claim 1, wherein the image detector comprises a photoacoustic transducer.

4. The catheter of claim 1, wherein the elongate member comprises a guidewire lumen.

5. The catheter of claim 4, wherein the image detector comprises an optical fiber mounted on an exterior surface of the guidewire lumen.

6. The catheter of claim 1, wherein the image detector comprises a fiber Bragg grating.

7. The catheter of claim 1, wherein the image detector receives the signal through the balloon as sound and sends the signal from the balloon to the proximal portion as light.

8. The catheter of claim 7, wherein the sound propagates substantially perpendicular to an axis of the catheter and the light propagates substantially parallel to the axis.

9. The catheter of claim 1, wherein the image detector comprises a fiber that is on an exterior of the elongate member within the balloon and entirely within the elongate member everywhere outside of the balloon.

10. The catheter of claim 1, wherein a flexibility of the catheter is substantially the same everywhere along a length of the distal portion outside of the balloon.

11. A method of delivering a balloon, the method comprising:
using an elongate catheter having a proximal portion and a distal portion to deliver a balloon disposed at the distal portion a treatment site within a vessel;
viewing the treatment site from within the balloon using an image detector within the balloon; and
inflating the balloon.

12. The method of claim 11, wherein the balloon further comprises a stent disposed thereon, and inflating the balloon deploys the stent.

13. The method of claim 11, wherein inflating the balloon causes an exterior surface of the balloon to make contact with the treatment site and dilate the vessel.

14. The method of claim 11, wherein viewing the treatment site comprises receiving an ultrasound image signal at the image detector.

15. The method of claim 14, wherein viewing the treatment site further comprises converting the ultrasound image signal into an optical interferometric signal using the image detector.

16. The method of claim 11, wherein the image detector comprises an optical fiber.

17. The method of claim 11, wherein the image detector comprises a fiber Bragg grating.

18. The method of claim 11, wherein the image detector comprises a blazed fiber Bragg grating.

19. The method of claim 11, wherein the image detector comprises a photoacoustic transducer.

20. The method of claim 11, wherein the image detector comprises a fiber Bragg grating, a blazed fiber Bragg grating, and a photoacoustic transducer.
